(19) United States
(12) Patent Application Publication
(10) Pub. No.: US 2025/0352475 A1
(43) Pub. Date: Nov. 20, 2025

(54) PROCESS FOR PREPARING HIGH-DENSITY COENZYME Q10 PARTICLES

(71) Applicant: TRADICHEM INDUSTRIAL SERVICES, S.L., ALCOBENDAS (ES)

(72) Inventors: José Ángel MARAÑÓN MAROTO, HOYO DE MANZANARES (ES); Patricia MORENO PUENTE, MADRID (ES); Cristina LOZANO MARTÍN, RIVAS VACIAMADRID (ES); Carlos RAMíREZ BORREGO, MADRID (ES)

(73) Assignee: TRADICHEM INDUSTRIAL SERVICES, S.L., ALCOBENDAS (ES)

(21) Appl. No.: 18/663,391

(22) Filed: May 14, 2024

(51) Int. Cl. A61K 9/16; A61K 31/122

(52) U.S. Cl. CPC A61K 9/1688; A61K 31/122

(57) ABSTRACT

The invention relates to a process for preparing compacted coenzyme Q10 particles of high density and improved flowability comprising roller compaction of coenzyme Q10 at a temperature comprised between 10° C. and 16° C., at a roll speed in the range 18-22 r.p.m., at a linear roll pressure in the range 20-30 kN/cm and with a gap between the rollers of 0.9 mm to 1.2 mm. Said process allows substantially increase the production capacity of highly-flowable compacted coenzyme Q10. The invention also relates to the compacted coenzyme Q10 obtainable with such process and to its use for preparing pharmaceutical compositions. The invention also relates to pharmaceutical compositions comprising the compacted coenzyme Q10, and to their therapeutic and cosmetic use.

### TECHNICAL FIELD

The present invention relates to an improved process for preparing compacted high-density coenzyme Q10 particles, which have improved flowability and handling properties.

### TECHNICAL BACKGROUND

Coenzyme Q10 (CoQ10) is an endogenous, vitamin-like benzoquinone, which has an important role in electron transport in mitochondrial membranes for the production of ATP and which also functions as an endogenous antioxidant.

It has been disclosed that some individuals suffer from CoQ10 deficiency, for example, due to genetic or aging factors, carcinogenesis processes or caused by statin treatment. In this connection, there are numerous diseases associated with CoQ10 deficiency which can benefit from the administration of coenzyme Q10, for example, cardiovascular diseases, neurodegenerative diseases, cancer, diabetes mellitus or male infertility, among others (Garrido-Maraver et al., Coenzyme Q10 Therapy, Mol. Syndromol., 2014, 187-197).

Furthermore, due to its anti-oxidant properties, CoQ10 is also frequently used in topical formulations in the cosmetic field, for reducing photoaging effects, for wrinkle reduction and for increasing epidermal turnover (Hoppe et al., Coenzyme Q10, a cutaneous antioxidant and energizer, BioFactors, 1999, 9, 371-378).

Therefore, compositions comprising CoQ10 as active ingredient are widely available, either as dietetic supplement, generally in form of capsules, softgels or tablets, or as topical formulation.

However, the preparation of formulations comprising CoQ10 is challenging due to the unfavourable physicochemical characteristics of this substance, more particularly, due to the instability and poor rheology of this substance. Indeed, coenzyme Q10 is a fine yellow to orange crystalline powder which is unstable and vulnerable to heat, light and oxygen. It decomposes and darkens when exposed to light, has low melting point, around 48° C., is highly hygroscopic and has poor flowability. Therefore, CoQ10 is difficult to be dosed accurately and pressed into tablets, and is difficult to handle as it easily adheres to machinery surfaces (Arenas-Jal et al., Coenzyme Q10 supplementation: Efficacy, safety, and formulation challenges, Compr. Rev. Food Sci. Food Saf., 2020, 19, 574-594).

Some solutions to these problems have been suggested in the art. The U.S. patent application US-A-2010/0004473 discloses a method for producing CoQ10 particles with improved handling and flowability. This method comprises mixing CoQ10 with a poor solvent, for example, aqueous ethanol solution or aqueous surfactant solution, previously heated at a temperature which is higher than the melting point of CoQ10, so that CoQ10 becomes melt and dispersed in the form of oil droplets, then the dispersion is cooled to below the solidification temperature of CoQ10, and the solid particles are then filtrated.

The European patent application EP-A-2415467 discloses a method for producing CoQ10 particles of improved fluidity and handling properties by compression molding of CoQ10 under specific pressure and temperature conditions, and then grinding the compressed fragment obtained. The temperature of CoQ10 during the compression must be in the range 35-52° C., and it is specifically stated that when the product temperature is lower than 35° C. a satisfactory compression molded product is not obtainable. Furthermore, for obtaining the best flowable product, having a low angle of repose of about 7 to 18 degrees, an additional step of heat treatment of the obtained powder is required, at a temperature in the range 30-52° C.

The European patent application EP-A-4101442 discloses a similar method for preparing compacted CoQ10, also by roller compaction. This method provides high-density CoQ10 having low angle of repose, without the need of a final step of heat treatment, by using lower temperature in the compression step. Accordingly, Examples 1 and 2 show that by performing the compression at a temperature in the range 20-25° C., using a linear pressure of 17.2-18.6 kN/cm and roll speed of 7-8 r.p.m., compacted coenzyme Q10 is obtained having 0.52 mg/ml bulk density and angle of repose value of 100.

However, the solutions provided so far in the prior art are inefficient, in terms of productivity and, consequently, are not optimal for their industrial implementation.

Therefore, there is still the need of simple and more efficient methods for producing CoQ10 particles of improved flowability.

### OBJECT OF THE INVENTION

The object of the present invention is a process for the preparation of compacted coenzyme Q10.

Another aspect of the invention is the compacted coenzyme Q10 obtainable by such process.

Another aspect of the invention is the use of said compacted coenzyme Q10 for the preparation of a pharmaceutical composition.

Another aspect of the invention is a pharmaceutical composition comprising the compacted coenzyme Q10.

Another aspect of the invention is said composition for use in therapy.

Another aspect of the invention the cosmetic use of said composition.

### DETAILED DESCRIPTION OF THE INVENTION

The object of the present invention is a process for preparing compacted coenzyme Q10 comprising roller compaction of coenzyme Q10, wherein the temperature is comprised between 10° C. and 16° C., the roll speed is comprised between 18 r.p.m. and 22 r.p.m., the roll force per unit of roller length is comprised between 20 kN/cm and 30 kN/cm, and the gap between the rollers is comprised between 0.9 mm and 1.2 mm.

The authors of the present invention have developed an improved process for preparing compacted coenzyme Q10, which not only provides a compacted, high-density coenzyme Q10 having improved flowability, but also, surprisingly, due to the multiple selection of specific process features, allows for much higher production capacity compared to the processes available in the prior art.

Along the present description, as well as in the claims, singular expressions, generally preceded by the articles “a”, “an” or “the”, are intended to include the plural forms as well, unless the context clearly indicates otherwise.

The terms “about” or “approximately” referred to amounts, as used herein, are meant to include the exact amount and also a certain deviation around the stated amount, namely of ±5%.

Unless otherwise indicated, the stated percentages are always weight percentages.

The numerical ranges disclosed herein are meant to include any number falling within the ranges and also the lower and upper limits.

Coenzyme Q10 Coenzyme Q10, frequently abbreviated as CoQ10, is a ubiquinone having a side chain of 10 isoprenoid units, also called ubidecarenone, ubiquinone 10 or ubiquinone 50 (Chemical name: 2-[(2E,6E,10E,14E,18E,22E,26E,30E,34E)-3,7,11,15,19,23,27,31,35,39-Decamethyltetraconta-2,6,10,14,18,22,26,30,34,38-decaenyl]-5,6-dimethoxy-3-methylcyclohexa-2,5-diene-1,4-dione, CAS number 303-98-0).

Non-compacted CoQ10, which is the starting material for the process of the present invention is typically produced by microbial fermentation (Lee et al., Cellular factories for coenzyme Q10 production, Microb. Cell Fact., 2017, 16, 39).

Furthermore, CoQ10 is widely available from different commercial sources.

Said non-compacted coenzyme Q10 starting material is typically in the form of fine particles, as a powder, namely, it is available as a yellow to orange crystalline powder. It has low melting point, ranging from 48° C. to 52° C., depending on the reporting source. It is hygroscopic, very adherent, and has poor flowability.

Its bulk (or apparent) density is generally less than 0.25 mg/ml, for example about 0.22 mg/ml. Its tapped (or compacted) density is typically less than 0.38 mg/ml, for example about 0.34 mg/ml.

Non-compacted, commercially available coenzyme Q10 used for the preparation of compacted coenzyme Q10, according to the process of the present invention, is typically a powder of fine particle size, with mean particle size generally of less than 800 microns, or less than 700 microns, or less than 600 microns, or less than 500 microns, or less 400 microns, or less than 300 microns, typically the mean particle size is in the range 100-300 microns.

CoQ10 from any source, either manufactured by any suitable method or obtained from any commercial source, can be used as starting material for the process of the present invention, provided that it has sufficient level of purity.

Non-compacted coenzyme Q10 used as starting material, as well as the compacted coenzyme Q10 obtained with the process of the invention, is substantially pure. That means that coenzyme Q10 has substantially no impurities and also that it is compacted alone, that is to say, it is not mixed with any other substance for performing the roller compaction process of the present invention, namely, it is not mixed with any excipient or vehicle.

Therefore, along the present description, as well as in the claims, both the non-compacted coenzyme Q10 used as starting material and the compacted coenzyme Q10 obtained with the process of the invention are always meant to be substantially pure, which typically means a purity of at least 95%, preferably of at least 96%, more preferably of at least 97%, still more preferably of at least 98%, and still more preferably of at least 98.5%. The purity can advantageously be even higher, of at least 99%, or of at least 99.5%, or about 100%. In one embodiment, the coenzyme Q10, both the starting material and the compacted product, fulfils the specifications of the pharmacopoeia of reference (e.g., 98.0% to 102.0% as stated in European Pharmacopoeia 10.0).

The purity degree of CoQ10 can be determined using methods known in the art, for example, by HPLC, for example, as disclosed in Lunetta et al., J AOAC Int., 2008, 91 (4), 702-8 or in the Ubidecarenone monograph of the European Pharmacopoeia 10.0.

### Compaction Process

Non-compacted, commercially available coenzyme Q10, as defined above, is used as starting material to prepare compacted coenzyme Q10, according to the process of the present invention.

The compaction process is performed using the roller compaction (or roll compaction) technology, which is well-known in the pharmaceutical field, namely, for the dry granulation of pharmaceutical powder mixtures. Such technology is widely described in different reference manuals in the art, for example, in R. W. Miller, Roller Compaction Technology, in: Handbook of Pharmaceutical Granulation Technology, Editor D. M. Parikh, Third Edition, Informa Healthcare USA, 2010, Chapter 8, 163-182. Briefly, in the roller compaction process, the powder being compacted is squeezed between two counter-rotating rolls to form a compressed sheet, which is subsequently milled into granules.

Roller compaction machines are well known in the art and are commercially available through many different companies, for example, Eurotab Bonals, Gerteis Maschinen+Processengineering, Freund Corporation, AlexanderWerk, Powtec, Hosokawa Alpine or L. B. Bohle Maschinen und Verfahrenmany, among many others.

As is well-known in the art, the roller compaction machines, or roller compactors, typically comprise a main roller unit connected with a feeding system, for feeding the starting material to be compressed into the roller unit, and are connected to a granulating/milling system, for milling the compacted sheet exiting from the rollers. “Roller” or “roll” terms are used interchangeably along the present description and in the claims.

The roller compactors available in the market may be characterized according to several parameters, for example, the type of feeding system, the range of compacting forces, the roll speed range, the diameter and the width (also referred to as the length) of the rolls, among others.

The roller unit consists of two equal diameter counter rotating rolls through which the powder is passed to get compacted. The two rolls can be mounted in horizontal, vertical or inclined position.

Typically, the roll diameter may range from about 100 to about 450 mm, depending on the roller compactor used. The roll diameter of the roller compactor to be used is not crucial, and all are suitable for performing the process of the current invention. For example, roll diameter of 150-250 mm is particularly suitable.

Typically, the roll width (also referred to as roll length) may range from about 20 to about 120 mm, and any width may be suitable for performing the method of the present invention. For example, widths comprised between 50 and 100 mm are suitable.

According to the present invention, some of the compression parameters must be specifically adjusted during the process, in particular, the temperature, the roll speed, the roll gap and the roll pressure.

The temperature during compression must be maintained between 10° C. and 16° C., preferably between 12° C. and 15° C.

Typically, the stated temperature during the compacting process is the temperature of the roller surface.

The temperature during compression can be kept under control by using any suitable refrigerating system and a temperature sensor. For example, the rollers may advantageously comprise an internal circuit with a circulating refrigerating fluid, for example water, whose flow can be regulated in order to adjust the temperature to the desired value, and typically comprising a temperature sensor, typically, on the roller surface. Generally, to maintain the temperature on the roller surface in the claimed range, i.e., between 10° C. and 16° C., the temperature of the refrigerating fluid, typically water, is in the range 6-9° C.

The temperature of the roller surface correlates well with the temperature of the product while it is being compressed between the rollers. Usually, the temperature of the product is a few degrees higher than the temperature in the roll surface.

Another parameter to be controlled is the roll speed. In particular, the roll speed must be adjusted to a value comprised between 18 r.p.m. and 22 r.p.m, preferably comprised between 19 r.p.m. and 21 r.p.m. and still more preferably, the roll speed is about 20 r. p. m.

Another parameter to be controlled is the roll gap, i.e., the distance between the two rolls. According to the process of the present invention, the roll gap must range from about 0.9 mm to about 1.2 mm, preferably from about 0.9 mm to about 1.1 mm, and more preferably the roll gap is about 1.0 mm.

Another parameter to be controlled is the compacting force. The roll force applied during the compression may be suitably expressed, for example, as force per unit of roller length (or linear pressure).

In the process according to the present invention, the force applied per unit of roller length is in the range from 20 kN/cm to 30 kN/cm. Preferably, the value is comprised between 23 kN/cm and 27 kN/cm, and more preferably is about 25 kN/cm.

The roller compactors available in the market may provide a wide range of compacting forces, generally ranging from about 10 kN to about 200 kN or higher.

Typically, the compacting force is comprised between 100 kN and 250 kN, preferably between 150 kN and 225 kN, and more preferably comprised between 175 kN and 200 kN.

The roll surface may be either smooth or non-smooth, i.e., may have rough pattern surface, for example, fluted, grooved or knurled surface. The roll surface of the two rolls may be identical or different.

In an embodiment of the invention, both rolls have smooth surface. In another embodiment of the invention, one roll has a smooth surface and the other has a non-smooth surface, for example, fluted, grooved or knurled surface.

Generally, one roll is fixed, while the other is movable and exerts pressure on the fixed roll by means of a hydraulic pressure control system.

As is well-known in the art, the feeding system in the roller compactor has the task of feeding the powder to be compacted to the rolls and ensures a uniform flow of material in order to continuously fill the nip between the rolls as they rotate. It can be a gravity feeder, wherein the powder is fed vertically by gravity, or a force feeder, wherein the powder is pushed towards the rolls by one or several screws. Preferably, screw feeding is used in the present invention, and the feeder can be vertical, horizontal or inclined. In one embodiment, the screw feeder is vertical and is situated in the upper part of the equipment, over the rolls. The screw feeder may be a hopper or analogous container containing an endless screw.

The feed speed can be adjusted, preferably to a constant feed speed, for example, by adjusting the rotation speed of the endless screw. Typically, the screw rotation speed is comprised between about 10 and about 50 r.p.m., preferably is comprised between 15 and 40 r.p.m., more preferably is comprised between 20 and 30 r.p.m., and still more preferably is about 25 r.p.m.

Typically, non-compacted coenzyme Q10 raw material is sent for compaction into the screw feeder from another hopper or reservoir containing the product, for example, using vacuum for sucking the powder and sending it to the screw feeder in a controlled way. The sucking of the raw material and its delivery into the screw feeder can be controlled in an automatic way, for example, by placing a probe inside the feeder that activates the feeding of the raw material when necessary.

Once coenzyme Q10 has been compacted, after passing through the two counter rotating rollers, it is transformed in a compressed sheet or ribbon, which is subsequently sent to the granulation/milling module, which is typically placed under the compacting rollers. Such granulation/milling module comprises one or more mills which break the compacted ribbon into compacted granules. Typically, after grinding, the granules are sieved through the suitable mesh size to obtain granules of the desired particle size. In this step of the compaction process, the speed of the granulation mill can also be adjusted. This speed is not critical, and is typically comprised in the range of from about 40 r.p.m. to about 500 r.p.m., preferably from about 50 r.p.m. to about 300 r.p.m., more preferably from about 60 r.p.m. to about 200 r.p.m, and still more preferably from about 70 to about 100 r.p.m.; for example, is selected from about 70 r.p.m. about 80 r.p.m., about 90 r.p.m., and about 100 r.p.m., more preferably is about 90 r.p.m.

In one embodiment of the invention, coenzyme Q10 is subjected to roller compaction using the following parameters:
temperature comprised between 10° C. and 16° C., preferably comprised between 12° C. and 15° C.;
roll speed comprised between 18 r.p.m. and 22 r.p.m., preferably comprised between 19 r.p.m. and 21 r.p.m., and still more preferably is about 20 r.p.m.;
roll gap comprised between 0.9 mm and 1.2 mm, preferably comprised between 0.9 mm and 1.1 mm, and more preferably is about 1.0 mm;
roll force per unit of roller length comprised between 20 kN/cm and 30 kN/cm, preferably comprised between 23 kN/cm and 27 kN/cm, and more preferably is about 25 kN/cm.; and/or force between the two rolls comprised between 100 kN and 250 kN, preferably comprised between 150 kN and 225 kN, and more preferably comprised between 175 kN and 200 kN;
   wherein preferably:
the screw rotating speed of the feeding system is comprised between 10 r.p.m. and 50 r.p.m., more preferably comprised between 15 r.p.m. and 40 r.p.m., still more preferably comprised between 20 r.p.m. and 30 r.p.m and still more preferably is about 25 r.p.m; and/or
the speed of the granulation mill is comprised between 50 r.p.m. and 300 r.p.m., preferably comprised between 60 r.p.m. and 200 r.p.m., more preferably comprised between 70 r.p.m. and 100 r.p.m., for example, is selected from about 70 r.p.m., about 80 r.p.m., about 90 r.p.m., and about 100 r.p.m., more preferably is about 90 r.p.m.

The compacted coenzyme Q10 obtainable with the process according to the present invention is in the form of granules having higher density and improved flowability compared to the commercial, non-compacted coenzyme Q10. As is well-known in the art, there are several parameters which can be used to assess the flowability of a powder or granulate, namely, the Carr index, Hausner ratio, flow rate and the angle of repose. Some of these parameters are related to the density of the substance, namely, to its bulk (apparent) density and its tapped (compacted) density.

The bulk density, also called apparent density, can be defined as the ratio of the mass to the volume of an untapped powder sample, as “poured”. The bulk density is given in grams per millilitre (g/ml) and is typically calculated by “pouring” a known mass of powder into a graduated cylinder.

On the other hand, tapped density, also called compacted density, of a powder is the ratio of the mass of the powder to the volume occupied by the powder after it has been tapped for a defined period of time. Tapped density is measured by first gently introducing a known sample mass into a graduated cylinder and carefully levelling the powder without compacting it. The cylinder is then mechanically tapped by raising the cylinder and allowing it to drop under its own weight using a suitable mechanical tapped density tester that provides a suitable fixed drop distance and nominal drop rate

Bulk density and tapped density can be measured according to the method disclosed in the European Pharmacopoeia 10.0 section 2.9.34 (Bulk density and tapped density of powders), for example, the bulk density may be measured using the PT-SV100 Scott Volumeter and the tapped density may be measured using the PT-TD200 Tap Density Tester (Pharma Test Apparatebau AG, Germany).

The Carr index, also called Carr's index or compressibility index, is calculated according to the following formula:

$$Carr index = \frac{D e nsity ( tapped ) - D e nsity ( bulk )}{D e nsity ( tapped )} \times 100$$

Free flowing powders have smaller values of the Carr index than poor flowing powders. In general, a Carr index greater than 25 is considered to be an indication of poor flowability, and below 15, of good flowability.

Hausner ratio is also predictive of powder flow, and is calculated according to the following formula:

$$Hausner ratio = \frac{D e nsity ( tapped )}{D e nsity ( bulk )} \times 100$$

In this case, a Hausner ratio greater than 1.34 is generally considered to be an indication of poor flowability.

The relationship between Carr index and/or Hausner ratio with powder flowability is well recognized in the art, for example, as disclosed by M. E. Aulton, in “Powder flow”, chapter 12 of the well-recognized reference book in pharmaceutical technology “Aulton's Pharmaceutics. The design and manufacture of medicines”, Fifth Edition 2018, M. E. Aulton and K. M. G. Taylor, Editors, Elsevier Ltd, as summarized in the following table:

| Type of flow | Carr index | Hausner ratio |
|---|---|---|
| Excellent | 1-10 | 1.00-1.11 |
| Good | 11-15 | 1.12-1.18 |
| Fair | 16-20 | 1.19-1.25 |
| Passable | 21-25 | 1.26-1.34 |
| Poor | 26-31 | 1.35-1.45 |
| Very poor | 32-37 | 1.46-1.59 |
| Very, very poor | >37 | >1.59 |

The “angle of repose” is another parameter for indirectly quantifying powder flowability. It is the angle at which a material will rest on a stationary heap, specifically, is the angle formed by the heap slope and the horizontal plane. It may be calculated by different methods, commonly, just pouring the material on a flat surface. It also can be calculated as “drained angle of repose”, as the angle measured on the internal conical face of a material which has been formed when drained from an orifice of a flat-bottomed container to the horizontal. A dynamic angle of repose can also be used, which is the angle to the horizontal of the free surface formed in a relatively slowly rotating drum (Aulton, op. cit., pg. 197).

In particular, the angle of repose can be measured according to the methods disclosed in the European Pharmacopoeia 10.0 section 2.9.36 (Powder flow).

In this case, the lower the value of the angle of repose, the higher the flowability of the material. It is generally considered that an angle of repose higher than 45 correlates with poor flowability, whereas an angle of repose under 30 correlates with excellent flowability.

As disclosed in Example 1, the compacted coenzyme Q10 obtained according to the process of the present invention has excellent flowability, as shown by the values of the Carr index, Hausner ratio and the angle of repose.

In particular, the method of the invention allows obtaining compacted CoQ10 having a Carr index lower than 15, preferably lower than 13, more preferably lower than 11, still more preferably equal to or lower than 10, for example comprised between 5 and 10.

Analogously, the method of the invention allows obtaining compacted CoQ10 having a Hausner ratio lower than 1.25, preferably lower than 1.20, more preferably lower than 1.18, still more preferably lower than 1.14, still more preferably lower than 1.12, and still more preferably equal to or lower than 1.11, namely, comprised between 1.00 and 1.11.

Non-compacted, commercially available coenzyme Q10, when subjected to the process according to the present invention, undergoes a significant increase of its density; typically, the bulk density increases in more than 100%, from a bulk density of about 0.2 g/ml of the starting non-compacted CoQ10 to a bulk density of more than 0.40 g/ml, preferably more than 0.45 g/ml and more preferably of more than 0.50 g/ml of the compacted CoQ10. Typically, the bulk density of the compacted CoQ10 is in the range 0.45-0.60 g/ml, preferably in the range 0.50-0.55 g/ml.

Furthermore, surprisingly, the authors of the present invention found that, by using a multiple combination of specific process parameters, namely, very low temperature, high pressure, high roller speed and high distance between rolls, it is possible maintain the good flow properties of the compacted coenzyme Q10 prepared according to the prior art document EP-A-4101442 but increasing in 233% the production. capacity, as shown in the Comparative Example.

The results are also surprising in view of the prior art document EP-A-2415467, where it is stated that the temperature of CoQ10 during the compression must be maintained in the range 35-52° C., and it is specifically stated that when the product temperature is lower than 35° C. a satisfactory compression molded product is not obtainable. Furthermore, it is stated in EP-A-2415467 that for obtaining the best flowable product, having a low angle of repose of about 7 to 18 degrees, an additional step of heat treatment of the compacted CoQ10 is required, at a temperature in the range 30-52° C.

The compacted CoQ10 obtained with the process of the present invention is advantageous for manufacturing pharmaceutical, dietary or cosmetic compositions due to its improved rheological properties, so it flows easily and does not stick to the walls of the containers during its handling and during the manufacturing process of said compositions, avoiding the need, for example, of using shaking or vibration. Furthermore, its improved compressibility and higher density make compacted CoQ10 particularly suitable as pharmaceutical, dietary or cosmetic ingredient.

Therefore, another aspect of the invention is substantially pure, compacted coenzyme Q10 which is obtainable by the process of the present invention, as disclosed above.

Remarkably, despite its known instability, after the compaction process of the present invention, coenzyme Q10 maintains its chemical integrity, so there is neither loss in purity nor increase in impurity occurrence. Therefore, the compaction process according to the present invention enables the preparation of substantially pure compacted CoQ10, with improved flowability characteristics, which has substantially the same purity degree as the starting material. The compacted CoQ10 obtained with the process of the invention is also substantially pure as defined above for the non-compacted CoQ10 starting material, and the purity can be determined in the same manner, typically by HPLC.

The size of the particles of compacted coenzyme Q10 can be adjusted, for example, by milling, if necessary, and sieving, using a sieve of suitable mesh size to obtain particles of the desired size, namely, under a specific size determined by the sieve opening. Typically, the mesh size of the sieve is adjusted to obtain particles of less than 2000 microns, preferably less than 1800 microns, preferably less than 1600 microns, more preferably less than 1400 microns, still more preferably less than 1200 microns, still more preferably less than 1000 microns, still more preferably less than 900 microns, and still more preferably less than 800 microns. The particle size, for example, is typically comprised between 400 and 2000 microns, preferably comprised between 400 and 1000 microns, and more preferably comprised between 500 and 800 microns.

### Pharmaceutical Compositions

As discussed above, the compacted coenzyme Q10 obtained with the process of the present invention, has improved flowability and higher density, and can be advantageously used for the preparation of pharmaceutical compositions.

Hence, the improved flowability is an advantage for the manufacturing of pharmaceutical compositions, as the product flows more easily, does not stick to the walls of vessels or blenders, or to the punches and dies of tableting machines, for example, therefore avoiding product loses and improving the equipment maintenance. Furthermore, the improved flowability of coenzyme Q10 is also an advantage for preparing powder pre-mixtures with excipients, for example, for manufacturing tablets or capsules, avoiding segregation of the components and providing better homogeneity of the blend.

Furthermore, the improved compressibility (related to its low Carr index) of compacted coenzyme Q10 particles enables the preparation of tablets of higher hardness by direct compression.

On the other hand, the higher density of compacted coenzyme Q10 prepared according to the process of the present invention enables a reduction of the volume of the final dosage forms, particularly, oral dosage forms such as capsules or tablets, for a given active ingredient strength.

Therefore, another aspect of the invention is the use of the compacted coenzyme Q10 of the present invention for the preparation of a pharmaceutical composition.

Another further aspect of the invention is a pharmaceutical composition comprising the compacted coenzyme Q10 of the present invention and at least one pharmaceutically acceptable excipient and/or vehicle.

Another aspect of the invention is a process for preparing a pharmaceutical composition comprising coenzyme Q10 and at least one pharmaceutically acceptable excipient and/or vehicle comprising a step of previously compacting substantially pure coenzyme Q10 alone, i.e., without any excipient or vehicle, by roller compaction, according to the process of the present invention. Subsequently, the pharmaceutical composition is prepared according to standard procedures.

Substantially pure compacted coenzyme Q10 is the active ingredient of the pharmaceutical compositions disclosed herein. Optionally, the composition can contain one or more additional active ingredients to reinforce the desired activity of the composition.

Typically, the pharmaceutical composition comprising substantially pure compacted CoQ10 as active ingredient, according to the present invention can be any conventional formulation which can be prepared using methods that are well known to the person skilled in pharmaceutical formulation, as those disclosed in handbooks of pharmaceutical technology, for example, in the book J. P Remington and A. R. Genaro, Remington The Science and Practice of Pharmacy, 20^{th} edition, Lippincott, Williams & Wilkins, Philadelphia, 2000 [ISBN: 0-683-306472] or in the book M. E. Aulton and K. M. G. Taylor, Aulton's Pharmaceutics, the design and manufacture of medicines, 5^{th} edition, Elsevier Ltd, 2018 [ISBN: 978-0-7020-7005-1], or in the book A. K. Dash, S. Singh and J. Tolman, Pharmaceutics. Basic principles and application to pharmacy practice, Academic Press, Elsevier, 2014 [ISBN: 978-0-12-386890-9].

The excipients suitable to be used in the pharmaceutical compositions of the present invention are also well known to those skilled in pharmaceutical technology and are described, for example, in the book R. C. Rowe, P. J. Sheskey and P. J. Weller, Handbook of Pharmaceutical Excipients, Sixth Edition, Pharmaceutical Press, 2009.

The term “pharmaceutical compositions”, are used herein, include not only oral, but also injectable and topical formulations.

Preferred are solid compositions, where the distinct properties of the compacted coenzyme Q10 granules according of the invention are particularly distinguishable. Therefore, in an embodiment of the invention, the pharmaceutical composition containing the compacted coenzyme Q10 is selected from the group consisting of tablets, powders, granules and capsules, wherein the capsules are preferably hard capsules, typically filled with powder or granulate material.

Furthermore, the term “pharmaceutical compositions” is applied herein to all compositions of CoQ10, irrespective of if they, for whatever reason, might be considered a dietary supplement or a cosmetic, rather than a medication. It is noted that, in general, the components and manufacturing processes of the formulations are substantially the same, irrespective of the fact that the final product can be classified either as a dietary supplement, a medication or a cosmetic composition. Therefore, along the present description, as well as in the claims, the term “pharmaceutical composition” may be replaced with either the term “cosmetic composition” or the term “dietary composition”, when the composition is intended for cosmetic use or may be classified as dietary supplement, respectively.

Oral pharmaceutical compositions may be solid, such as tablets, capsules, powders or granules; or liquid, such as solutions, suspensions or syrups.

In a preferred embodiment, the oral pharmaceutical composition is a solid pharmaceutical composition selected from tablets, capsules, powders and granules, preferably selected from tablets, hard capsules, powders and granules. Hard capsules are preferably filled with powder or granulate material.

Tablets comprising compacted coenzyme Q10 of the present invention can be formulated using standard procedures, well known in the art. Preferably, tablets are prepared by direct compression taking advantage of the good compressibility of compacted CoQ10, i.e., compacted CoQ10 is mixed with suitable excipients and compressed without any previous granulation process. Alternatively, compacted CoQ10 can be previously granulated with additional excipients, either by wet granulation, fluid-bed granulation or dry granulation, and then optionally mixed with extra-granular excipients and compressed into tablets. Excipients typically used in the formulation of tablets are diluents, binding agents, glidants, lubricants, disintegrating agents, and mixtures thereof. Other additional excipients include sweetening agents, flavouring agents and colouring agents, for example.

Suitable diluents include calcium carbonate, calcium phosphate, calcium sulfate, cellulose acetate, dextrates, dextrins, dextrose, ethylcellulose, fructose, glyceryl palmitostearate, isomalt, kaolin, lactitol, lactose, magnesium carbonate, magnesium oxide, maltodextrins, maltose, mannitol, microcrystalline or powdered cellulose, pregelatinized starch, starch, sodium carbonate, sodium chloride, sorbitol and sucrose, among others, and mixtures thereof.

Suitable binding agents include acacia, agar, cellulose acetate phthalate, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropylmethyl cellulose, methylcellulose, maltodextrin, microcrystalline cellulose, povidone, pregelatinized starch, sodium carboxymethylcellulose, starch, stearic acid and sucrose, among others, and mixtures thereof.

Suitable glidants include powdered cellulose, colloidal silicon dioxide, magnesium oxide, magnesium silicate, magnesium trisilicate, silicon dioxide, and talc, among others, and mixtures thereof.

Suitable lubricants include calcium stearate, glyceryl behenate, glyceryl palmitostearate, hydrogenated castor oil, magnesium stearate, myristic acid, palmitic acid, polyethylene glycol, sodium benzoate, sodium lauryl sulphate, sodium stearyl fumarate, stearic acid, and talc, among others, and mixtures thereof.

Suitable disintegrants include alginic acid, crospovidone, sodium croscarmellose, sodium starch glycolate, starch, and low-substituted hydroxypropyl cellulose, among others, and mixtures thereof.

Furthermore, tablets may be formulated either as conventional compressed tablets, or in other forms, for example, buccal, sublingual, chewable, sustained-release or orally-disintegrating tablets, by selecting suitable excipients and manufacturing processes, as are well known in the art.

Capsules, as is well known in the pharmaceutical field, are solid dosage forms in which the active substance is enclosed in either a hard or soft, soluble container or shell. The raw materials used in the manufacture of the shell are similar for both hard and soft gelatin capsules, while the proportions vary. The major component of a capsule shell is typically gelatin; other components include water, plasticizers, such as glycerol, sorbitol or propylene glycol, colorants, opacifiers, such as titanium dioxide, and preservatives. Hypromellose can alternatively be used as capsule shell material for hard capsules.

Hard capsules consist of two sections, one slipping over the other and thus enclosing the drug formulation, which is generally in solid form, as powder or granulate, or occasionally in liquid or semi-liquid form, while in soft gelatin capsules, also referred to as softgels, a continuous, one-piece, hermetically sealed gelatin shell surrounds the drug formulation, which is generally in liquid, suspension or semisolid form.

The powders or granules included within hard capsules comprise the active ingredient and typically at least one pharmaceutically acceptable excipient, generally selected from diluents, lubricants and glidants, and mixtures thereof, which are well-known in the art, for example, those disclosed above for the formulation of tablets.

The term “powders” typically refers to intimate mixtures of finely divided active ingredient, optionally with one or more excipients. The active ingredient and the excipient(s) are mixed to obtain a homogeneous mixture, for example using trituration, spatulation, sifting or tumbling procedures, which are well-known in the art.

The term “granules” typically refers to larger particles, either containing the active ingredient alone, or, more frequently, aggregations of active ingredient particles with additional excipients, typically prepared by dry granulation, wet granulation or fluid-bed granulation procedures, which are also well known in the art.

The fill compositions for soft gelatin capsules generally comprise the active ingredient, i.e., CoQ10, dissolved or dispersed in a liquid vehicle. The vehicle is typically an oily liquid, for example, a vegetable oil, such as peanut oil, castor oil, olive oil, rapeseed oil, corn oil, sesame oil, sunflower oil or soybean oil, among others, or medium chain triglycerides (MCT), or mixtures thereof. Other liquid, non-oily, vehicles can also be used, for example, polyethylene glycols, isopropyl alcohol, propylene glycol, glycerol, polyglycerols, triacetin, glyceryl esters, sorbitan esters, sugar esters and polyglyceryl esters, among others, or mixtures thereof. The composition generally additionally comprises other excipients, acting as suspending agents and/or viscosity modifying agents, for example, hydrogenated vegetable oils, waxes, or ethylcellulose for oily vehicles and solid glycol esters for non-oily vehicles. Surfactants such as lecithin or polysorbate 80 may also be added to improve the dispersion of the suspended drug particles. Alternatively, the fill composition may be in the form of self-emulsifying lipophilic systems or microemulsion pre-concentrates, which typically comprise a lipophilic solvent, such as a vegetable oil or a medium chain triglyceride, and a surfactant, and optionally a co-solvent, such as ethanol or propyleneglycol.

Powders or granules for oral administration may be directly administered to the oral cavity to be swallowed or either they can be previously dissolved or dispersed in water or in other suitable liquid before being ingested. They may also be effervescent powders or granules.

The formulation and preparation of powders or granules for oral administration is analogous to the formulation and preparation of powders or granules for preparing tablets or for filling capsules, as discussed above. They typically comprise the active ingredient, i.e., CoQ10, and at least one pharmaceutically acceptable excipient, generally selected from diluents, lubricants, glidants, binders, disintegrating agents, and mixtures thereof, which are well-known in the art, for example, those disclosed above for the formulation of tablets. They may contain also additional excipients, such as colouring and flavouring substances. Furthermore, as is well-known in the art, effervescent powders additionally contain acid substances, for example, citric acid, and carbonates or hydrogen carbonates which react rapidly in the presence of water and effervesce releasing carbon dioxide.

Powders or granules for oral administration are presented as single-dose or multi-dose preparations in suitable containers, for example, bulk containers provided with a measuring device for multi-dose preparations, or mono-dose sachets for single-dose preparations. Those sachets can be made of paper or either of aluminium or plastic laminates.

In an embodiment, the pharmaceutical composition comprising compacted coenzyme Q10 of the present invention and at least one pharmaceutically acceptable excipient is a solid form for oral administration selected from the group consisting of tablets, hard capsules, soft capsules, powders and granules, and preferably selected from tablets, hard capsules filled with powder o granulate material, powders and granules.

Suitable liquid oral dosage forms typically include solutions, suspensions and syrups.

Oral solutions contain CoQ10 active substance dissolved in a vehicle, while syrups are oral aqueous solutions containing high concentrations of sucrose or other sugars. Sugar-free syrups are obtained by replacing sucrose with hydrogenated glucose, mannitol, sorbitol or xylitol, for example.

As coenzyme Q10 is poorly soluble in water, the formulation strategies for formulating oral solutions or syrups include, for example, using water in combination with co-solvents, using a solubilizer, and/or encapsulating coenzyme Q10 in the form of liposomes using suitable phospholipids, among others.

Oral suspensions contain coenzyme Q10 dispersed, rather than dissolved, in a liquid medium.

Suitable excipients included in solutions and/or suspension are, for example, solubilizers, stabilizers, buffers, antioxidants, preservatives, flavouring agents, colouring agents, and sweetening agents, for example.

Suitable co-solvents include ethanol, propylene glycol, polyethylene glycol 300 or 400, sorbitol and glycerol, among others, and mixtures thereof.

Suitable solubilizing agents include cyclodextrins and surfactants, such as sorbitan esters, polyoxyethylene castor oil derivatives, polysorbates, sodium oleate, potassium oleate, or polyoxyethylene stearate, among others, and mixtures thereof.

Suitable preservatives include ascorbic acid, sorbic acid, benzalkonium chloride, benzyl alcohol, bronopol, parabens, sodium benzoate, sodium propionate, or thimerosal, among others, and mixtures thereof.

Suitable antioxidants include ascorbic acid, sodium sulphite, sodium bisulphite and sodium metabisulfite, among others, and mixtures thereof.

Suitable viscosity modifiers include acacia, alginic acid, bentonite, carbomers, carrageenan, gelatin, glycerol, hydroxyethyl cellulose, hydroxyethylmethyl cellulose, hydroxypropyl cellulose, maltodextrin, polyvinyl alcohol, sodium alginate, tragacanth, xanthan gum, gellan gum, guar gum, among others and mixtures thereof.

Suitable suspending agents include xanthan gum, guar gum, alginic acid, bentonite, carbomers, sodium or calcium carboxymethylcellulose, hydroxyethyl cellulose, hydroxypropyl cellulose, hydroxypropylmethyl cellulose, hydroxypropyl alginate, microcrystalline or powdered cellulose, anhydrous colloidal silica, dextrins, gelatins, kaolin, magnesium aluminium silicate, maltitol, povidone, sorbitan esters, or tragacanth, among others, and mixtures thereof.

Suitable flavouring agents include maltol, vanillin, ethyl vanillin, menthol, citric acid, fumaric acid, ethyl maltol, tartaric acid, peppermint, artificial or natural fruit aromas, among others, and mixtures thereof.

Suitable sweetening agents include sorbitol, maltitol, mannitol, dextrose, isomalt, maltose, xylitol, saccharin, sucrose, sucralose, aspartame, acesulfame potassium or trehalose, among others, and mixtures thereof.

Typically, such liquid dosage forms for oral use may be supplied as multi-dose or as single-dose preparations. Each dose from a multi-dose container is generally administered using a device suitable for measuring the prescribed volume. The measuring device may be, for example, a spoon, a cup, an oral syringe, or a dropper.

In an embodiment, the pharmaceutical composition comprising compacted coenzyme Q10 of the present invention and at least one pharmaceutically acceptable excipient is a liquid formulation for oral administration selected from the group consisting of solutions, suspensions and syrups.

The compacted coenzyme Q10 of the present invention can also be formulated as a parenteral composition, namely, for intravenous or intramuscular administration. Parenteral or injectable compositions, as is well-known in the art, are sterile formulations in the form of solution, emulsion or suspension, prepared by dissolution, emulsification or suspension of compacted coenzyme Q10 active ingredient in water for injection, or, alternatively, in other vehicles. Alternatively, the injectable composition may be in the form of powder, for preparing the solution immediately before the administration by dissolving the powder in water for injection. As disclosed above, due to the low water solubility of CoQ10, solubilizer substances may be needed in such formulations, for example, surfactants, such as fatty acid esters of polyoxyethylene or sorbitan, among others, as well as co-solvents, such as ethyl alcohol, glycerol, polyethylene glycol, or propylene glycol, among others, and mixtures thereof.

Other suitable excipients that can be added to parenteral formulations include antimicrobials, such as benzalkonium chloride, benzyl alcohol, chlorobutanol, metacresol, parabens, phenol or thimerosal, among others, and mixtures thereof; antioxidants, such as ascorbic acid, cysteine, monothioglycerol, sodium bisulfite, sodium metabisulfite, tocopherols, among others, and mixtures thereof; buffers, such as acetates, citrates or phosphates, among others; chelating agents, such as salts of EDTA; and tonicity-adjusting agents, such as dextrose or sodium chloride, among others, or mixtures thereof; among others, and mixtures thereof.

In an embodiment, the pharmaceutical composition comprising compacted CoQ10 of the present invention and at least one pharmaceutically acceptable excipient is an injectable composition.

Compacted coenzyme Q10 can also be formulated as a topical composition, namely, in liquid or semisolid form. Liquid topical compositions include lotions, liniments and tinctures, and they are typically prepared by dissolving or dispersing CoQ10 in a suitable carrier such as, for example, water, alcohols, glycols, or mixtures thereof, optionally including some solubilizing aid, as disclosed above. Topical liquid compositions may be directly applied on the area of skin or mucosal tissue to be treated or, alternatively, they can be used to impregnate a dressing or bandage to be applied on the treated area.

Semisolid topical compositions include creams, gels, ointments and pastes. Typically, they are prepared by dissolving or suspending CoQ10 in a suitable pharmaceutically acceptable carrier. This carrier is selected from water, a non-aqueous water miscible carrier, such as for example ethanol or isopropanol, and a non-aqueous water-immiscible carrier, such as for example paraffin oil. Preferably, semisolid topical compositions for CoQ10 are in the form of creams. As is well-known in the art, creams are semisolid emulsions, which are classified as oil-in-water (o/w) and water-in-oil (w/o) types depending on whether the continuous phase is oil or water. Typically, such semisolid compositions for topical administration contain a pharmaceutically acceptable excipient such as, for example, surfactant and emulsifier agents, lipidic and emollient compounds, consistency factors and thickening agents, stabilizers, hydrotropes, preservative agents, essences, colouring agents, silicone compounds, fats, waxes, lecithins, phospholipids, UV sun protection factors, or mixtures thereof.

Suitable topical compositions also include ophthalmic solutions, which are intended for application to the conjunctiva, conjunctival sac or eyelids and may be in the form of aqueous solutions, suspensions, emulsions, or reconstitutable powders. The formulation of ophthalmic compositions is well known in the art, and common excipients employed include the vehicle, which is typically water for injection, buffering agents, preservatives, tonicity agents, viscosity modifiers, surfactants and stabilizers.

In an embodiment, the pharmaceutical composition comprising compacted CoQ10 of the present invention and at least one pharmaceutically acceptable excipient is a topical composition selected from the group consisting of lotions, liniments, tinctures, creams, gels, ointments, pastes, and ophthalmic solutions, preferably selected from lotions, creams and ophthalmic solutions.

The dose of coenzyme Q10 contained in each unit dose may widely vary depending on the route of administration and the therapeutic or cosmetic indication. The skilled in the art can easily adjust the most suitable amount of coenzyme Q10 for each type of composition and use.

For example, in oral dosage forms, each unit dose typically comprises from about 10 to about 1000 mg, preferably from about 100 to about 500 mg of coenzyme Q10.

### Use

Another aspect of the present invention relates to a pharmaceutical composition comprising the compacted coenzyme Q10 of the present invention for use in therapy.

Another aspect of the invention is a method of treating a disease or condition susceptible to treatment with coenzyme Q10 in a subject, the method comprising administering a pharmaceutical composition comprising the compacted coenzyme Q10 of the present invention to the subject.

The terms “treatment”, “treating” or “therapy” as used herein have their conventional meaning and typically refer to the administration of the pharmaceutical composition with the purpose of attenuating, alleviating, minimising, inhibiting the progression, or suppressing the symptoms of the treated disease or condition, or for stimulating the recovery after said disease or condition. Furthermore, the treatment is also meant to include the preventive aspect, i.e., the administration of the coenzyme Q10 containing pharmaceutical composition to a subject suspected or prone to suffer from a specific disease or condition in order to prevent the onset of such disease or condition. The pharmaceutical composition comprising compacted coenzyme Q10 for use in therapy, according to this aspect of the invention, is meant therefore to include the treatment and/or prevention of any disease or condition susceptible to be treated or prevented with CoQ10.

Among the possible therapeutic indications of coenzyme Q10 are, for example, human CoQ10 deficiencies, mitochondrial diseases, fibromyalgia, cardiac failure, ischemic heart disease, interaction with statins, hypertension, endothelian function, diabetes, cancer, Parkinson's disease, Huntington's disease, Alzheimer's disease, Friedreich's ataxia, asthenozoospermia, periodontal disease, migraine, pre-eclampsia and Down's syndrome, among others (Garrido-Maraver et.al., op. cit. or Coenzyme Q10 in: Braun L. and Cohen M., Elsevier, 2017, Essential Herbs & Natural Supplements, ISBN: 9780729542685, pg. 276-309).

The therapeutically effective dose of coenzyme Q10 widely varies depending on the therapeutic indication, the route of administration, the severity of the condition, and the age of the patient. The recommended dose may typically range from about 50 to about 1200 mg/day (see, for example, Braun L. and Cohen M op. cit.). The expert practitioner will have no difficulty in selecting the most appropriate dosage in each case.

Another aspect of the invention is the cosmetic use of a composition comprising compacted coenzyme Q10.

The “cosmetic use” is related to beautify and/or improve the appearance of normal, non-diseased skin. Coenzyme Q10 may be used as a cosmetic ingredient, generally in topical formulations, for example, for reducing photoaging effects, for wrinkle reduction and for increasing epidermal turnover (Hoppe et al., op. cit.)

Either for use in therapy or as cosmetic, coenzyme Q10 may be administered alone, i.e., as the only active ingredient of the composition, or combined with other active ingredients, either in the same composition, as a fixed-dose combination, or in combination with other pharmaceutical/cosmetic/dietetic formulations.

### Examples

### Example 1.—Preparation of Compacted coQ10 According to the Process of the Present Invention

For the preparation of compacted high-density coenzyme Q10, commercially available coenzyme Q10 (Inner Mongolia Kingdomway Pharmaceutical Ltd) was used. The coenzyme Q10 raw material was a yellow-orange crystalline powder, of bulk density 0.22 g/ml and particle size of less than 800 microns.

The purity of coenzyme Q10 before and after the compaction process was assessed by means of HPLC. The HPLC specifications used were as follows:
Equipment: 1260 Infinity LC (Agilent Technologies).
Column: ACE3 C18 PFP (150×4.6 mm)
Flow rate: 1 ml/min
Injection volume: 10 μl
Detector: 275 nm
Run time: 10 minutes
Solvent: ethanol:methanol (20:80). Isocratic elution
Retention time: 5.25

Both the original coenzyme Q10 and the compacted coenzyme Q10 obtained after the compaction process had chemical purity greater than 99%, determined by HPLC.

For the compaction process, a roller compactor equipment was used (Bonals BC-150/75V), with vacuum system for feeding the raw material into the screw feeding system and with a refrigerating system. The roller compactor had one roll with smooth surface and one roll with knurled surface. The dimensions of both rolls were 10.5 cm diameter and 7.4 cm width.

The roll speed in the roller compactor was set to 20 r.p.m. Screw feeding speed was 25 r.p.m., roll force was set to 185 kN (25 kN/cm) and the roll gap was fixed to 1.0 mm.

The roller compactor used allowed a continuous control of the temperature of the roll surface and had an internal refrigeration circuit, with a circulating water at a temperature of about 8° C. The temperature in the roller surface was kept in the range 12-15° C. during the compression process.

The compactor was connected to a rotor granulator for milling the outcoming compacted plaque. The granulation speed was fixed to 90 r.p.m. and the obtained granulate was passed through a sieve of 800 micron opening.

The particles of compacted coenzyme Q10 had a size of less than 800 micron.

The process as above described allowed for a production capacity of 100 Kg/h of compacted coenzyme Q10.

The obtained compacted coenzyme Q10 was in the form of free-flowing, non-agglomerated orange granules. Its flow parameters are shown in the following table, and are compared to non-compacted coenzyme Q10 starting material:

| Property | Non-compacted CoQ10 | Compacted CoQ10 |
|---|---|---|
| Bulk density | 0.22 g/ml | 0.51 g/ml |
| Tapped density | 0.34 g/ml | 0.56 g/ml |
| Carr Index | 35.3% | 8.92% |
| Hausner ratio | 1.55 | 1.09 |
| Angle of repose | 45° | 12° |

The bulk density was measured using a PT-SV100 Scott Volumeter (Pharma Test) and the tapped density was measured using a PT-TD200 Tap Density Tester (Pharma Test).

The angle of repose was determined by measuring the radius and the height of the cone formed after pouring the powder, using the formula tan⁻¹ (height/radius), according to the method disclosed in the European Pharmacopoeia 10.0 section 2.9.36 (Powder flow).

### Comparative Example Preparation of Compacted coQ10 According to the Prior Art

Compacted coenzyme Q10 was prepared according to the process disclosed in EP-A-4101442.

The starting material was the same coenzyme Q10 raw material used in Example 1 (Inner Mongolia Kingdomway Pharmaceutical Ltd).

As in Example 1, the purity of coenzyme Q10 before and after the compaction process was assessed by means of HPLC, with the same specifications disclosed above.

It was found that both the original coenzyme Q10 and the compacted coenzyme Q10 obtained after the compaction process had chemical purity greater than 99%, determined by HPLC.

The same roller compactor equipment disclosed in Example 1 was used.

The roll speed in the roller compactor was set to 8 r.p.m. Screw feeding speed was adjusted to 15 r.p.m.

The roll force per was kept in the range 127.5-137.3 kN (17.2-18.6 kN/cm). The roll gap was fixed to 0.5 mm.

The temperature was kept in the range 20-25° C. during the compression process.

The granulation speed was fixed to 70 r.p.m. and the obtained granulate was passed through a sieve of 800 micron opening.

The production capacity of the above process was 30 Kg/h of compacted coenzyme Q10, much lower compared to the process of the present invention (233% increase).

The obtained compacted coenzyme Q10 were also free-flowing, non-agglomerated orange granules. The following table summarizes the flow parameters of the obtained compacted coenzyme Q10:

| Property | Compacted CoQ10 |
|---|---|
| Bulk density | 0.52 g/ml |
| Tapped density | 0.57 g/ml |
| Carr Index | 8.78% |
| Hausner ratio | 1.10 |
| Angle of repose | 10° |

### Example 2.—Preparation of Capsules with the Compacted Coenzyme Q10 of Example 1

Coenzyme Q10 hard gelatin capsules of 100 mg strength were prepared using the ingredients listed in the following table:

| Ingredient | Amount per capsule |
|---|---|
| Compacted CoQ10 (Example 1) | 100 mg |
| Maize starch (diluent) | 200 mg |
| Magnesium stearate (lubricant) | 10 mg |

All ingredients were added to a Turbula mixer, blended during 15 minutes, and filled into capsules of 00 size.

The capsules were easily prepared, it was observed that the compacted CoQ10 used had good flowability and did not adhere to the walls of vessels and mixers. Furthermore, the powder mixture was easily blended and good homogeneity was easily achieved, without segregation of the ingredients.

### Example 3.—Preparation of Tablets by Direct Compression with the Compacted Coenzyme Q10 of Example 1

Coenzyme Q10 tablets of 30 mg strength were prepared using the ingredients listed in the following table:

| Ingredient | Amount per tablet | |
|---|---|---|
| Compacted CoQ10 (Example 1) | 30 | mg |
| Calcium phosphate (diluent) | 75 | mg |
| Microcrystalline cellulose (diluent) | 100 | mg |
| Magnesium stearate (lubricant) | 2 | mg |
| Silica (glidant) | 2 | mg |

All ingredients were mixed in Turbula mixer during about 15 minutes and compressed into tablets.

It was also observed that the compacted CoQ10 used had good flowability and did not adhere neither to the walls of vessels and mixers nor to the punches and dies of the tableting machine. The powder mixture was easily blended and had good homogeneity, without segregation of the ingredients.

1. A process for preparing compacted coenzyme Q10 comprising roller compaction of coenzyme Q10, wherein the temperature is comprised between 10° C. and 16° C., the roll speed is comprised between 18 r.p.m. and 22 r.p.m., the roll force per unit of roller length is comprised between 20 kN/cm and 30 kN/cm, and the gap between the rollers is comprised between 0.9 mm and 1.2 mm.

2. The process according to claim 1, wherein the temperature is comprised between 12° C. and 15° C.

3. The process according to claim 1, wherein the roll speed is comprised between 19 r.p.m. and 21 r.p.m.

4. The process according to claim 1, wherein the gap between the rollers is comprised between 0.9 mm and 1.1 mm.

5. The process according to claim 1, wherein the roll force per unit of roller length is comprised between 23 kN/cm and 27 kN/cm.

6. The process according to claim 5, wherein the roll force per unit of roller length is about 25 kN/cm.

7. The process according to claim 1, wherein the roll force is comprised between 100 kN and 250 kN, preferably comprised between 150 kN and 225 kN, and more preferably comprised between 175 kN and 200 kN.

8. A compacted coenzyme Q10 obtainable by a process according to claim 1.

9. The compacted coenzyme Q10 according to claim 8, wherein it is in the form of granules having a particle size comprised between 400 and 2000 microns, preferably comprised between 500 and 800 microns.

10. A method of preparing a pharmaceutical composition, comprising using compacted coenzyme Q10 according to claim 8.

11. A pharmaceutical composition comprising compacted coenzyme Q10 according to claim 8 and at least one pharmaceutically acceptable excipient and/or vehicle.

12. The pharmaceutical composition according to claim 11, characterized in that it is a solid oral pharmaceutical composition selected from tablets, capsules, powders and granules.

13. The pharmaceutical composition according to claim 11 for use in therapy.

14. A cosmetic comprising a composition according to claim 11.
